SEQUENCE LISTING (11 sequences)

SEQ ID NO: 1 - protein, 9 aa, Artificial Sequence
  wound-homing sequence
CARSKNKDC

SEQ ID NO: 2 - DNA, 35 bp, Artificial Sequence
  primer WIF-1 NheI fwd
tcagtcgctagcgccaccatggctcggagaagagc

SEQ ID NO: 3 - DNA, 33 bp, Artificial Sequence
  primer WIF-1 BsrGI rev
tcagtctgtacagggttcaccagatgtaattgg

SEQ ID NO: 4 - DNA, 25 bp, Artificial Sequence
  primer WIF1 (fwd)
ttctttaaaacatgtcaacaagctg

SEQ ID NO: 5 - DNA, 20 bp, Artificial Sequence
  primer WIF1 (rev)
acaaaagcctccatttcgac

SEQ ID NO: 6 - DNA, 20 bp, Artificial Sequence
  primer HPRT (fwd)
gtcaacgggggacataaaag

SEQ ID NO: 7 - DNA, 22 bp, Artificial Sequence
  primer HPRT (rev)
tgcattgttttaccagtgtcaa

SEQ ID NO: 8 - DNA, 20 bp, Artificial Sequence
  primer IL1beta (fwd)
tcgtgctgtctgacccatgt

SEQ ID NO: 9 - DNA, 24 bp, Artificial Sequence
  primer IL1beta (rev)
acaaagctcatggagaataccact

SEQ ID NO: 10 - DNA, 24 bp, Artificial Sequence
  primer IL6 (fwd)
aactccatctgcccttcaggaaca

SEQ ID NO: 11 - DNA, 24 bp, Artificial Sequence
  primer IL6 (rev)
aaggcagtggctgtcaacaacatc

(19) United States
(12) Patent Application Publication
(10) Pub. No.: US 2019/0275035 A1
(43) Pub. Date: Sep. 12, 2019

(54) WNT ANTAGONISTS AND THEIR USE IN METHODS FOR TREATING MYOCARDIAL INFARCTION

(71) Applicant: RUPRECHT-KARLS-UNIVERSITÄT HEIDELBERG, Heidelberg (DE)

(72) Inventors: Sören MEYER, Heidelberg (DE); Florian LEUSCHNER, Heidelberg (DE); Hugo KATUS, Heidelberg (DE)

(73) Assignee: RUPRECHT-KARLS-UNIVERSITÄT HEIDELBERG, Heidelberg (DE)

(21) Appl. No.: 16/347,216

(22) PCT Filed: Nov. 6, 2017
(86) PCT No.: PCT/EP2017/078266

§ 371 Date: May 3, 2019

(30) Foreign Application Priority Data
Nov. 4, 2016 (EP) 16197263.3

(51) Int. Cl. A61K 31/497; C12N 15/86; A61K 38/17; A61P 11/10; C07K 14/47; C07K 14/475; A61K 48/00

(52) U.S. Cl. CPC A61K 31/497; C12N 15/86; A61K 38/17; A61K 48/0066; C07K 14/4703; C07K 14/475; A61K 48/0058; A61P 11/10

(57) ABSTRACT

The present invention relates to the treatment of myocardial infarction, especially to the reduction of tissue damage, the reduction of infarction scars, the improvement of cardiac function, and/or the prevention of congestive heart failure after myocardial infarction. The invention further relates to WNT antagonists, their use in the treatment of myocardial infarction, and to pharmaceutical compositions comprising WNT antagonists.

### FIELD OF THE INVENTION

The present invention relates to the treatment of myocardial infarction, especially to the reduction of tissue damage, the reduction of infarction scars, the improvement of cardiac function, and/or the prevention of congestive heart failure after myocardial infarction. The invention further relates to WNT antagonists, their use in the treatment of myocardial infarction, and to pharmaceutical compositions comprising WNT antagonists.

### BACKGROUND OF THE INVENTION

Myocardial infarction (MI) is one of the leading causes of death in industrialized nations. Although improved treatment has tremendously increased survival of acute MI, the incidence of subsequent heart failure remains high. Our understanding of inflammatory processes following cardiac injury has progressed in recent years. We now know that an adequate inflammatory response is crucial for the healing process after cardiac injury, but prolonged and exaggerated (or diminished) inflammation can cause additional tissue damage and promote adverse remodeling (Saxena et al. 2016). Monocytes and monocyte-derived macrophages are critical players not only in the development of atherosclerosis and coronary heart disease but also in the immune response to cardiac ischemia (Geissmann et al. 2010). Following coronary occlusion and subsequent myocardial damage, neutrophils are the first dominant leukocyte subset to invade the infarcted heart. Their numbers peak early, during the first days after cardiac injury, and monocytes and their lineage-descendant macrophages become the predominant infiltrating cell types over the course of the first week (Swirski et al. 2013). The monocyte/macrophage response is biphasic: whereas pro-inflammatory Ly-6C^{hi} monocytes dominate the early phase (1-4 days post injury), reparative Ly-6C^{lo} macrophages are the predominant cell type at later stages (Nahrendorf et al. 2007; Hilgendorf et al. 2014; He et al. 2015). The inflammatory Ly-6C^{hi} monocytes are highly proteolytic and phagocytotic. They clear dead cellular debris and damaged extracellular matrix from the infarcted area. The reparative Ly-6C^{lo} monocytes/macrophages promote angiogenesis and collagen deposition to form a solid scar and replace the lost heart tissue (Swirski et al. 2013). Balancing monocytes/macrophages' pro- and anti-inflammatory properties seems to be a prerequisite for optimal cardiac healing. Going forward, we need further insights into the signals controlling this balanced response, especially since clinical anti-inflammatory strategies have failed thus far (Christia et al. 2013).

Recent research has revealed that WNT signaling has immunomodulating properties and is induced by inflammatory mediators (Pereira et al. 2008; Kim et al. 2012; Rauner et al. 2012). WNT signaling can be characterized as either the β-catenin-dependent canonical WNT pathway or the β-catenin-independent non-canonical WNT/PCP pathway, which uses c-Jun N-terminal kinases (JNK), among others, for signal transduction. The canonical WNT signaling cascade is essential for normal cardiogenesis (Eisenberg et al. 2006). WNT signaling in the adult heart, however, is silenced but reactivated after cardiac injury (Koval et al. 2011). There is an increasing body of evidence that reactivating the canonical WNT pathway negatively affects infarct healing with respect to cardiomyocyte death and cardiac fibrosis (Daskalopoulos et al. 2013). Yet the effects of modulating the non-canonical WNT pathway, in the context of myocardial healing, have barely been studied.

Modulating WNT signaling often occurs extracellularly via several WNT antagonists, which can be divided into two functional groups that both prevent ligand-receptor interactions. Members of the first group, Dickkopf (DKK), bind to WNT receptors and inhibit signal transduction, and the second group, secreted Frizzled-related protein (sFRP), bind directly to WNT proteins and block them from binding to their receptors (Kawano et al. 2003; Clevers et al. 2012). The WNT Inhibitory Factor 1 (WIF1) binds directly to WNT proteins (Hsieh et al. 1999). To date, WIF1 has not been studied in the context of either myocardial infarction or inflammation.

### TECHNICAL PROBLEMS UNDERLYING THE PRESENT INVENTION

Current therapies of myocardial infarction (MI) focus on the treatment of coronary occlusion, i.e. on the reperfusion of the blocked vessel. However, MI is often associated with inflammation processes that can cause additional tissue damage and may ultimately lead to congestive heart failure.

Until now, there are no therapies that successfully modulate the inflammation processes occurring after MI.

### SUMMARY OF THE INVENTION

The inventors of the present invention have now studied the influence of the local microenvironment on the infarcted myocardium and monocyte activation. The inventors surprisingly found that WIF1 plays a key role during monocyte activation by controlling the inflammatory process after cardiac injury, thereby affecting cardiac function.

The inventors have identified the WNT signaling pathway as a potential target for the modulation of the inflammation processes associated with MI. The inventors have also demonstrated in vivo that WNT antagonists are suitable for the treatment of MI, especially for reducing tissue damage, for reducing infarction scars, for improving cardiac function, and/or for preventing congestive heart failure after myocardial infarction.

Accordingly, in a first aspect the present invention relates to a WNT antagonist for use in the treatment of myocardial infarction.

In a second aspect the present invention relates to a fusion protein comprising:

(a) WNT inhibitory factor 1 (WIF1), and
(b) a wound-homing sequence.

In a third aspect the present invention relates to an expression vector comprising a polynucleotide sequence encoding a protein that antagonizes the non-canonical WNT signalling pathway.

In a fourth aspect the present invention relates to a pharmaceutical composition comprising a WNT antagonist and further comprising one or more compounds selected from the group consisting of a pharmaceutically acceptable carrier, diluent, excipient, filler, binder, lubricant, glidant, disintegrant, adsorbent, and preservative.

This summary of the invention does not necessarily describe all features of the present invention. Other embodiments will become apparent from a review of the ensuing detailed description.

### BRIEF DESCRIPTION OF THE FIGURES

FIG. 1. Myocardial infarction produces differential gene expression profiles in inflammatory monocytes sorted from the bone marrow and heart.

FIG. 1A. Log₂(x-fold) of canonical WNT pathway inhibitors in Ly6C^{hi} monocytes sorted from the heart compared to Ly6C^{hi} monocytes in the bone marrow.

FIG. 1B. Log₂(x-fold) of non-canonical WNT/PCP pathway mediators in Ly6C^{hi} monocytes sorted from the heart compared to Ly6C^{hi} monocytes in the bone marrow.

FIG. 2. Non-canonical WNT increases following MI and is inhibited by WIF1. Quantification of pJNK expression (mean±SD, N=6, *P≤0.05). C).

FIG. 3. WIF1 protein expression during MI.

FIG. 3A. WNT inhibitor mRNA levels in isolated neonatal rat cardiomyocytes cultured under hypoxic conditions. Results from three independent experiments performed in triplicate (mean±SD, *P≤0.05).

FIG. 3B. WIF1 mRNA levels in isolated neonatal rat cardiac fibroblasts cultured under hypoxic conditions. Results originate from three independent experiments performed in triplicate (mean±SD, P>0.05).

FIG. 3C. Quantification of WIF1 protein expression in sham-operated and LAD-ligated animals. (mean±SD, N=4, *P≤0.01).

FIG. 4. Global WIF1 knockout worsens MI outcome.

FIG. 4A. Quantification of relative scar size in WIF1KO and their WT littermates four weeks after MI.

FIG. 4B. Heart weight/body weight ratios four weeks after induced MI (mean±SD, N=11, *P≤0.02)

FIG. 4C. Echocardiographic results from WIF1KO and their WT littermates four weeks after MI (mean±SD, N=11, *P≤0.02).

FIG. 4D. Quantification of total neutrophils following MI (P=0.91).

FIG. 4E. cTNT levels one day after LAD ligation (mean±SD, N=11, P=0.65).

FIG. 4F. Quantification of total cell numbers per mg heart tissue of inflammatory (Ly6C^{hi}) monocytes (top left, *P≤0.04); Ly6C^{hi} monocytes per μl blood (top, right); reparative) (Ly6C^{lo}) macrophages per mg heart tissue (bottom left, P≤0.05) and Ly6C^{hi} monocytes per femur (bottom right). Results are represented as mean±SD with N=8-11 per group.

FIG. 5. Cardiac-specific AAV-9-mediated WIF1 overexpression improves heart function following MI.

FIG. 5A. Timeline of AAV-mediated WIF1 overexpression experiments.

FIG. 5B. cTNT levels one day after LAD ligation (mean±SD, N=6, P=0.19.)

FIG. 5C. Heart weight/body weight ratios four weeks after induced MI (mean±SD, N=6, *P≤0.04).

FIG. 5D. Quantification of relative scar size in AAV-WIF1 and AAV-LUC control animals four weeks after MI (mean±SD, N=5, *P≤0.03).

FIG. 5E. Echocardiographic results from AAV9-LUC-injected and AAV9-WIF1-injected mice four weeks after MI (mean±SD, N=6, *P≤0.01).

FIG. 5F. Quantification of inflammatory (Ly6C^{hi}) monocytes (left graph, *P≤0.05) and reparative)(Ly6C^{lo}) macrophages (right graph, P=0.58) per mg heart tissue. Results are represented as mean±SD with N=12 per group.

FIG. 6. WIF1 inhibits non-canonical WNT signaling.

FIG. 6A. Quantification of pJNK expression in macrophages stimulated with supernatant of AdWIF1-transfected hypoxic cardiomyocytes (mean±SD, N=3-4, *P≤0.05).

FIG. 6B. mRNA levels of inflammatory markers in macrophages stimulated with supernatant of control or WIF1 overexpressing cardiomyocytes cultured under hypoxic conditions (mean±SD, N=3, *P≤0.05).

FIG. 7. LGK-974 inhibits WNT signaling in stressed/hypoxic cardiac muscle cells.

FIG. 7A. Representative western blots of Active-beta-catenin (ABC), cleaved caspase-3 and GAPDH of isolated neonatal rat ventricular cardiomyocytes (NRVCM) cultured under hypoxic or normoxic conditions treated with LGK-974 or vehicle control.

FIG. 7B. Quantification of western blots. Expression of ABC and cleaved caspase-3 were normalized to GAPDH expression.

FIG. 8. LGK-974 reduces monocyte inflammatory processes after myocardial infarction in a mouse model.

FIG. 8A. Comparison of troponin T levels in LGK-974-treated animals vs. control animals (mean±SD, P=0.67).

FIG. 8B. FACS analysis: Cell count of leukocytes (CD45+) 2 days after I/R surgery in control animals.

FIG. 8C. FACS analysis: Cell count of leukocytes (CD45+) 2 days after I/R surgery in LGK-974-treated animals.

FIG. 8D. Quantification of leukocytes (CD45+) per mg heart tissue in LGK-974-treated animals vs. control animals (mean±SD, P=0.059).

FIG. 8E. Quantification of inflammatory monocytes (CD45+; Lineage−; CD11b+; CD11c−; LY6C+) per mg heart tissue in LGK-974-treated animals vs. control animals (mean±SD, *P≤0.05).

### DETAILED DESCRIPTION OF THE INVENTION

### Definitions

Before the present invention is described in detail below, it is to be understood that this invention is not limited to the particular methodology, protocols and reagents described herein as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art to which this invention belongs.

Preferably, the terms used herein are defined as described in “A multilingual glossary of biotechnological terms: (IUPAC Recommendations)”, Leuenberger, H. G. W, Nagel, B. and Kölbl, H. eds. (1995), Helvetica Chimica Acta, CH-4010 Basel, Switzerland).

Throughout this specification and the claims which follow, unless the context requires otherwise, the word “comprise”, and variations such as “comprises” and “comprising”, will be understood to imply the inclusion of a stated member, integer or step or group of members, integers or steps but not the exclusion of any other member, integer or step or group of members, integers or steps.

Several documents (for example: patents, patent applications, scientific publications, manufacturer's specifications, instructions, GenBank Accession Number sequence submissions etc.) are cited throughout the text of this specification. Nothing herein is to be construed as an admission that the invention is not entitled to antedate such disclosure by virtue of prior invention. Some of the documents cited herein are characterized as being “incorporated by reference”. In the event of a conflict between the definitions or teachings of such incorporated references and definitions or teachings recited in the present specification, the text of the present specification takes precedence.

Sequences: All sequences referred to herein are disclosed in the attached sequence listing that, with its whole content and disclosure, is a part of this specification.

As used herein, the term “WNT antagonist” refers to any compound that is capable of inhibiting the WNT signaling pathway, in particular the non-canonical WNT signaling pathway. This inhibition can occur at various levels of the WNT signaling pathway. For example, the WNT antagonist can inhibit synthesis of WNT proteins (e.g. on the transcription level or the translation level), can directly or indirectly inhibit secretion of WNT proteins, or can inhibit binding between WNT proteins and one or more WNT receptors.

LGK-974 (chemical name: 2-[5-methyl-6-(2-methylpyridin-4-yl)pyridin-3-yl]-N-(5-pyrazin-2-ylpyridin-2-yl)acetamide) is a potent and selective inhibitor of Porcn that can prevent secretion of WNT proteins (Liu J. et al, 2013, Proc. Natl. Acad. Sci. U.S.A., 110(50):20224-9).

WNT inhibitory factor 1 is a protein that in humans is encoded by the WIF1 gene. The protein WIF1 is a lipid-binding protein that binds to WNT proteins and prevents them from triggering signalling. The WIF1 protein contains a WNT inhibitory factor (WIF) domain and 5 epidermal growth factor (EGF)-like domains. It may be involved in mesoderm segmentation. This protein is found to be present in fish, amphibia and mammals.

The human WIF1 protein is a protein of 379 amino acids, including a signal peptide spanning amino acids 1 to 28. Thus, the mature protein spans amino acids 29-379. The amino sequence of WIF1 has the NCBI Reference Sequence number: NP_009122.2. The amino acid sequence of human WIF1 can also be retrieved from the UniProt database via accession number Q9Y5W5.

As used herein, the expression “mature WIF1 protein” refers to a polypeptide consisting of amino acids 29-379 of NCBI entry NP_009122.2.

In the context of the present invention and unless clearly otherwise specified, the expressions “WNT inhibitory factor 1” and its abbreviation “WIF1” encompass the mature WIF1 protein (as defined above) as well as muteins of the mature WIF1 protein, wherein said muteins of the mature WIF1 protein exhibit at least 90% amino acid sequence identity (e.g. at least 91%, at least 92%, at least 93%, at least 94%, at least 95%, at least 96%, at least 97%, at least 98% and at least 99% sequence identity) to said mature WIF1 protein and are capable of binding to WNT proteins and preventing WNT proteins from signalling.

The term “amino acid sequence identity” refers to a quantitative comparison of the identity (or differences) of the amino acid sequences of two or more proteins. “Percent (%) amino acid sequence identity” with respect to a reference polypeptide sequence is defined as the percentage of amino acid residues in a sequence that are identical with the amino acid residues in the reference polypeptide sequence, after aligning the sequences and introducing gaps, if necessary, to achieve the maximum percent sequence identity.

To determine the sequence identity, the sequence of a query protein is aligned to the sequence of a reference protein. Methods for alignment are well-known in the art. For example, for determining the extent of an amino acid sequence identity of an arbitrary polypeptide relative to a reference amino acid sequence, the SIM Local similarity program is preferably employed (Xiaoquin Huang and Webb Miller (1991), Advances in Applied Mathematics, vol. 12: 337-357), that is freely available (see also: http://www.expasy.org/tools/sim-prot.html). For multiple alignment analysis ClustalW is preferably used (Thompson et al. (1994) Nucleic Acids Res., 22(22): 4673-4680). Preferably, the default parameters of the SIM Local similarity program or of ClustalW are used, when calculating sequence identity percentages.

In the context of the present invention, the extent of sequence identity between a modified sequence and the sequence from which it is derived is generally calculated with respect to the total length of the unmodified sequence, if not explicitly stated otherwise.

In embodiments, where neither sequence is a modified sequence, the extent of sequence identity is calculated with respect to the total length of the reference sequence. For example, in the expression “protein A exhibits at least 90% amino acid sequence identity to protein B”, the amino acid sequence of protein B is the reference sequence, and the extent of sequence identity is calculated with respect to the total length of sequence B. This applies in a fully analogous manner to nucleotide sequences. For example, in the expression “nucleic acid A exhibits at least 90% sequence identity to nucleic acid B”, the nucleotide sequence of nucleic acid B is the reference sequence, and the extent of sequence identity is calculated with respect to the total length of sequence B.

Each amino acid of the query sequence that differs from the reference amino acid sequence at a given position is counted as one difference. The sum of differences is then related to the length of the reference sequence to yield a percentage of non-identity. The quantitative percentage of identity is calculated as 100 minus the percentage of non-identity.

These explanations apply in a fully analogous manner to nucleotide sequences. Thus, each nucleotide of the query sequence that differs from the reference nucleic acid sequence at a given position is counted as one difference. The sum of differences is then related to the length of the reference sequence to yield a percentage of non-identity. The quantitative percentage of identity is calculated as 100 minus the percentage of non-identity.

The “muteins of the mature WIF1 protein” preferably differ from the “mature WIF1 protein” by one or more amino acid substitutions, more preferably by conservative substitutions. In some embodiments of the invention, the “muteins of the mature WIF1 protein” differ from the “mature WIF1 protein” by 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 amino acid substitutions, preferably by conservative substitutions.

“Conservative substitutions” may be made, for instance, on the basis of similarity in polarity, charge, size, solubility, hydrophobicity, hydrophilicity, and/or the amphipathic nature of the amino acid residues involved. Amino acids can be grouped into the following six standard amino acid groups:

(1) hydrophobic: Met, Ala, Val, Leu, Ile;

(2) neutral hydrophilic: Cys, Ser, Thr, Asn, Gln;

(3) acidic: Asp, Glu;

(4) basic: His, Lys, Arg;

(5) residues that influence chain orientation: Gly, Pro; and

(6) aromatic: Trp, Tyr, Phe.

As used herein, “conservative substitutions” are defined as exchanges of an amino acid by another amino acid listed within the same group of the six standard amino acid groups shown above. For example, the exchange of Asp by Glu retains one negative charge in the so modified polypeptide. In addition, glycine and proline may be substituted for one another based on their ability to disrupt α-helices. Some preferred conservative substitutions within the above six groups are exchanges within the following sub-groups: (i) Ala, Val, Leu and Ile; (ii) Ser and Thr; (ii) Asn and Gln; (iv) Lys and Arg; and (v) Tyr and Phe. Given the known genetic code, and recombinant and synthetic DNA techniques, the skilled scientist readily can construct DNAs encoding the conservative amino acid variants.

As used herein, “non-conservative substitutions” or “non-conservative amino acid exchanges” are defined as exchanges of an amino acid by another amino acid listed in a different group of the six standard amino acid groups (1) to (6) shown above.

The term “fusion protein” relates to a protein comprising at least a first protein joined genetically to at least a second protein. A fusion protein is created through joining of two or more genes that originally coded for separate proteins. Thus, a fusion protein may comprise a multimer of identical or different proteins which are expressed as a single, linear polypeptide.

As used herein, a “small molecule” refers to an organic molecule with a molecular weight of 2000 g/mol or less, preferably with a molecular weight of 1000 g/mol or less.

As used herein, a “patient” means any mammal or bird who may benefit from a treatment with a WNT antagonist described herein. Preferably, a “patient” is selected from the group consisting of laboratory animals (e.g. mouse or rat), domestic animals (including e.g. guinea pig, rabbit, chicken, turkey, pig, sheep, goat, camel, cow, horse, donkey, cat, or dog), or primates including monkeys (e.g. African green monkeys, chimpanzees, bonobos, gorillas) and human beings. It is particularly preferred that the “patient” is a human being. The terms “patient” and “subject to be treated” (or in short: “subject”) are used interchangeably herein.

As used herein, “treat”, “treating” or “treatment” of a disease or disorder means accomplishing one or more of the following: (a) reducing the severity and/or duration of the disorder; (b) limiting or preventing development of symptoms characteristic of the disorder(s) being treated; (c) inhibiting worsening of symptoms characteristic of the disorder(s) being treated; (d) limiting or preventing recurrence of the disorder(s) in patients that have previously had the disorder(s); (e) limiting or preventing recurrence of symptoms in patients that were previously symptomatic for the disorder(s); (f) reduction of mortality after occurrence of a disease or a disorder; (g) healing; and (h) prophylaxis of a disease.

As used herein, “prevent”, “preventing”, “prevention”, or “prophylaxis” of a disease or disorder means preventing that a disorder occurs in a subject for a certain amount of time. For example, if a WNT antagonist described herein is administered to a subject with the aim of preventing a disease or disorder, said disease or disorder is prevented from occurring at least on the day of administration and preferably also on one or more days (e.g. on 1 to 30 days; or on 2 to 28 days; or on 3 to 21 days; or on 4 to 14 days; or on 5 to 10 days) or for one or more months (e.g. for 1 to 36 months, or 2 to 30 months, or 3 to 24 months, or 4 to 18 months, or 5 to 12 months or 6 to 9 months) following the day of administration.

A “pharmaceutical composition” according to the invention may be present in the form of a composition, wherein the different active ingredients and diluents and/or carriers are admixed with each other, or may take the form of a combined preparation, where the active ingredients are present in partially or totally distinct form. An example for such a combination or combined preparation is a kit-of-parts.

The term “active ingredient” refers to the substance in a pharmaceutical composition or formulation that is biologically active, i.e. that provides pharmaceutical value. In the context of the invention, the active ingredient is a WNT antagonist as defined in the first aspect and/or a fusion protein of the second aspect and/or an expression vector of the third aspect. A pharmaceutical composition may comprise one or more active ingredients which may act in conjunction with or independently of each other. The active ingredient can be formulated as neutral or salt forms. The salt form is preferably a pharmaceutically acceptable salt. The active ingredient can be administered to a cell, a tissue or an individual in an effective amount.

An “effective amount” is an amount of a therapeutic agent sufficient to achieve the intended purpose. The effective amount of a given therapeutic agent will vary with factors such as the nature of the agent, the route of administration, the size and species of the subject to receive the therapeutic agent, and the purpose of the administration. The effective amount in each individual case may be determined empirically by a skilled person according to established methods in the art. The expressions “effective amount” and “therapeutic amount” are used interchangeably herein. If the context does not state otherwise, the term “therapeutic agent” refers to the WNT antagonists of the invention.

“Pharmaceutically acceptable” means approved by a regulatory agency of the Federal or a state government or listed in the U.S. Pharmacopeia or other generally recognized pharmacopeia for use in animals, and more particularly in humans.

The term “carrier”, as used herein, refers to a diluent, adjuvant, excipient, or vehicle with which the therapeutic agent is administered. Such pharmaceutical carriers can be sterile liquids, such as saline solutions in water and oils, including those of petroleum, animal, vegetable or synthetic origin, such as peanut oil, soybean oil, mineral oil, sesame oil and the like. A saline solution is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsions, tablets, pills, capsules, powders, sustained-release formulations and the like. The composition can be formulated as a suppository, with traditional binders and carriers such as triglycerides. The compounds of the invention can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc. Examples of suitable pharmaceutical carriers are described in “Remington's Pharmaceutical Sciences” by E. W. Martin. Such compositions will contain a therapeutically effective amount of the compound, preferably in purified form, together with a suitable amount of carrier so as to provide the form for proper administration to the patient. The formulation should suit the mode of administration.

As used in the context of the invention, “administering” includes in vivo administration to an individual as well as administration directly to cells or tissue in vitro or ex vivo. In a preferred embodiment of the invention, the pharmaceutical compositions are customized for the treatment of a disease or disorder.

In a particularly preferred embodiment of the invention, a treatment with a pharmaceutical composition according to the invention comprises the treatment of an individual after myocardial infarction and the healing comprises the improvement of left ventricular systolic function and may be associated with an increase in capillary density in the infarct border zone. Additionally, it may reduce the mortality after a myocardial infarction. The methods which can be used to determine parameters like the improvement of left ventricular systolic function, increase in capillary density in the infarct border zone and the reduction of mortality after a myocardial infarction are well known in the art.

The pharmaceutical composition contemplated by the present invention may be formulated in various ways well known to one of skill in the art. For example, the pharmaceutical composition of the present invention may be in liquid form such as in the form of solutions, emulsions, or suspensions. Preferably, the pharmaceutical composition of the present invention is formulated for parenteral administration, preferably for intravenous, intraarterial, intramuscular, subcutaneous, transdermal, intrapulmonary, intraperitoneal intracoronary, intracardiac administration, or administration via mucous membranes, preferably for intravenous, subcutaneous, or intraperitoneal administration. A preparation for oral or anal administration is also possible. Preferably, the pharmaceutical composition of the present invention is in the form of a sterile aqueous solution which may contain other substances, for example, enough salts or glucose to make the solution isotonic with blood. The aqueous solutions should be suitably buffered (preferably to a pH of from 3 to 9, more preferably to a pH of from 5 to 7), if necessary. The pharmaceutical composition is preferably in unit dosage form. In such form the pharmaceutical composition is subdivided into unit doses containing appropriate quantities of the active component. The unit dosage form can be a packaged preparation, the package containing discrete quantities of pharmaceutical composition such as vials or ampoules.

The administration of the pharmaceutical composition is preferably administered through the intravenous, intraarterial, intramuscular, subcutaneous, transdermal, intrapulmonary, intraperitoneal, intracoronary or intracardiac route wherein other routes of administration known in the art are also comprised.

In the case that the pharmaceutical composition is used as a treatment for an individual, the use of the pharmaceutical composition can replace the standard treatment for the respective disease or condition or can be administered additionally to the standard treatment. In the case of an additionally use of the pharmaceutical composition, the pharmaceutical composition can be administered before, simultaneously or after a standard therapy. In a preferred embodiment, the standard therapy is a reperfusion therapy and the pharmaceutical composition can be administered before, simultaneously or after the reperfusion therapy.

It is further preferred that the pharmaceutical composition is administered once or more than once. This comprises 2, 3, 4, 5, 6, 7, 8, 9, 10, 11, 12, 13, 14, 15, 16, 17, 18, 19, 20, 25, 30, 35, 40, 45 or 50 times. The time span for the administration of the pharmaceutical is not limited. Preferably, the administration does not exceed 1, 2, 3, 4, 5, 6, 7, 8, 9, 10, 11, or 12 weeks. A single dose of the pharmaceutical composition, can independently from the overall amount of administered doses or the respective time span of administration be administered as one or more bolus injection(s) and/or infusion(s).

### Embodiments of the Invention

The present invention will now be further described. In the following passages different aspects of the invention are defined in more detail. Each aspect defined below may be combined with any other aspect or aspects unless clearly indicated to the contrary. In particular, any feature indicated as being preferred or advantageous may be combined with any other feature or features indicated as being preferred or advantageous.

In a first aspect the present invention is directed to a WNT antagonist for use in the treatment of myocardial infarction.

In an alternative wording, the first aspect of the present invention is directed to the use of a WNT antagonist in the preparation of a pharmaceutical composition for the treatment of myocardial infarction.

In another alternative wording, the first aspect of the present invention is directed to a method for the treatment of myocardial infarction in a subject, comprising the step of administering a therapeutic amount of a WNT antagonist to a subject in need thereof.

In an embodiment of the first aspect, treatment of myocardial infarction comprises:

(i) reducing tissue damage after myocardial infarction, and/or
(ii) reducing infarction scars after myocardial infarction, and/or
(iii) improving cardiac function after myocardial infarction, in particular improving left ventricular function, and/or
(iv) preventing congestive heart failure after myocardial infarction.

In an embodiment of the first aspect, the WNT antagonist

inhibits secretion of WNT,

blocks binding of WNT to one or more WNT receptors, and/or

inhibits production of WNT.

In embodiments, in which the WNT antagonist blocks binding of WNT to one or more WNT receptors, said blocking is effected by binding of the WNT antagonist to WNT, or by binding of the WNT antagonist to said one or more WNT receptors, or both.

In an embodiment of the first aspect, the WNT antagonist is

(a) a small molecule; e.g. LGK-974 (i.e. 2-[5-methyl-6-(2-methylpyridin-4-yl)pyridin-3-yl]-N-(5-pyrazin-2-ylpyridin-2-yl)acetamide).
(b) an expression vector comprising a polynucleotide sequence encoding a protein that antagonizes the non-canonical WNT signalling pathway, or
(c) a protein; e.g. WIF1 or a fusion protein comprising WIF1.

In some embodiments, the WNT antagonist is an expression vector as defined above in paragraph (b) and said expression vector is an AAV vector, preferably an AAV9 vector. In some of these embodiments, the polynucleotide sequence is under the control of a cardiomyocyte-specific promoter, such as the troponin T promoter. In further embodiments, the protein that antagonizes the non-canonical WNT signalling pathway is WNT inhibitory factor 1 (WIF1) or a fusion protein comprising WIF1.

In other embodiments, the WNT antagonist is a fusion protein comprising WIF1 as defined above in paragraph (c). In some of these embodiments, said fusion protein comprises a wound-homing sequence. In a preferred embodiment, the wound-homing sequence is CARSKNKDC (SEQ ID NO: 1). In some embodiments, the wound-homing sequence can be fused directly to WIF1; in other embodiments, the fusion protein additionally comprises a peptide linker (e.g. a linking amino acid sequence of 1 to 20 amino acids) that connects the wound-homing sequence to WIF1.

In a second aspect the present invention is directed to a fusion protein comprising:

(a) WNT inhibitory factor 1 (WIF1), and
(b) a wound-homing sequence.

In some embodiments of the second aspect, the wound-homing sequence is CARSKNKDC (SEQ ID NO: 1).

In some embodiments, the wound-homing sequence can be fused directly to WIF1; in other embodiments, the fusion protein additionally comprises a peptide linker (e.g. a linking amino acid sequence of 1 to 20 amino acids) that connects the wound-homing sequence to WIF1.

In a third aspect the present invention is directed to an expression vector comprising a polynucleotide sequence encoding a protein that antagonizes the non-canonical WNT signalling pathway.

In some embodiments of the third aspect, the expression vector is an AAV vector, preferably an AAV9 vector.

In some embodiments of the third aspect, the polynucleotide sequence is under the control of a cardiomyocyte-specific promoter, e.g. the troponin T promoter.

In some embodiments of the third aspect, the protein that antagonizes the non-canonical WNT signalling pathway is WNT inhibitory factor 1 (WIF1) or a fusion protein comprising WIF1. In further embodiments the fusion protein comprises a wound-homing sequence, e.g. the wound-homing sequence is CARSKNKDC (SEQ ID NO: 1). In some embodiments, the wound-homing sequence can be fused directly to WIF1; in other embodiments, the fusion protein additionally comprises a peptide linker (e.g. a linking amino acid sequence of 1 to 20 amino acids) that connects the wound-homing sequence to WIF1.

In a fourth aspect the present invention is directed to a pharmaceutical composition comprising a WNT antagonist and further comprising one or more compounds selected from the group consisting of a pharmaceutically acceptable carrier, diluent, excipient, filler, binder, lubricant, glidant, disintegrant, adsorbent, and preservative.

In an embodiment of the fourth aspect, the WNT antagonist

inhibits (or is capable of inhibiting) secretion of WNT,

blocks (or is capable of blocking) binding of WNT to one or more WNT receptors, and/or

inhibits (or is capable of inhibiting) production of WNT.

In embodiments, in which the WNT antagonist blocks binding of WNT to one or more WNT receptors, said blocking is effected by binding of the WNT antagonist to WNT, or by binding of the WNT antagonist to said one or more WNT receptors, or both.

In an embodiment of the fourth aspect, the WNT antagonist is

(a) a small molecule; e.g. LGK-974 (i.e. 2-[5-methyl-6-(2-methylpyridin-4-yl)pyridin-3-yl]-N-(5-pyrazin-2-ylpyridin-2-yl)acetamide).
(b) an expression vector comprising a polynucleotide sequence encoding a protein that antagonizes the non-canonical WNT signalling pathway, or
(c) a protein; e.g. WIF1 or a fusion protein comprising WIF1.

In some embodiments of the fourth aspect, the WNT antagonist is an expression vector as defined above in paragraph (b) and said expression vector is an AAV vector, preferably an AAV9 vector. In some of these embodiments, the polynucleotide sequence is under the control of a cardiomyocyte-specific promoter, such as the troponin T promoter. In further embodiments, the protein that antagonizes the non-canonical WNT signalling pathway is WNT inhibitory factor 1 (WIF1) or a fusion protein comprising WIF1.

In other embodiments of the fourth aspect, the WNT antagonist is a fusion protein comprising WIF1 as defined above in paragraph (c). In some of these embodiments, said fusion protein comprises a wound-homing sequence. In a preferred embodiment, the wound-homing sequence is CARSKNKDC (SEQ ID NO: 1). In some embodiments, the wound-homing sequence can be fused directly to WIF1; in other embodiments, the fusion protein additionally comprises a peptide linker (e.g. a linking amino acid sequence of 1 to 20 amino acids) that connects the wound-homing sequence to WIF1.

In particularly preferred embodiments of the fourth aspect, the WNT antagonist is a fusion protein as defined in the second aspect or an expression vector as defined in the third aspect.

### Examples

The following Examples are provided for further illustration of the invention. The invention, however, is not limited thereto, and the following Examples merely show the practicability of the invention on the basis of the above description.

### 1. Methods

### 1.1 Animals

WIF1 KO mice were provided by Dr. Igor B. Dawid (National Institutes of Health, Bethesda, USA). WIF1 KO were generated by inserting a tau-LacZ reporter cassette into the WIF1 coding sequence (Kansara M 2009). Animals were backcrossed for at least 6 generations and maintained on C57BL6 background (Janvier, Saint-Berthevin, France). The procedure used male KO animals and their WT littermates, age 10-12 weeks. Cardiac-specific WIF1 overexpression in 6-week-old male C57BL6 (Janvier, Saint-Berthevin, France) was reached by i.v. injection of 10¹² AAV particles (for details on AAV production, see below). WIF1-overexpression was established for four weeks. Animals were housed under standard laboratory conditions with a 12-hour light-dark cycle and access to water and food ad libitum. All experimental protocols were approved by the institutional review board of the University of Heidelberg, Germany, and the responsible government authority of Baden-Württemberg, Germany (project number 35-9185.81/G-27/14).

### 1.2 Induction of Myocardial Infarction

Myocardial infarction was induced by permanent ligation of the left anterior descending (LAD) coronary artery in male mice aged 10-12 weeks. In brief, anesthesia was induced with isoflurane (4%/800 ml O₂/min) and maintained by endotracheal ventilation (2-3%/800 ml O₂/min). Thoracotomy was performed in the fourth left intercostal space. The left ventricle was exposed, and the left coronary artery was permanently occluded. Chest and skin were closed, and anesthesia was terminated. Animals were extubated when breathing was restored. Initial myocardial injury was evaluated by measuring cardiac Troponin T levels in plasma 24 hours after induction of myocardial infarction.

### 1.3 Echocardiography

Echocardiography was performed on a Visualsonic Vevo 2100 four weeks after induction of myocardial infarction. Mice were conscious during echocardiographic measurements. Ejection fraction (EF) and fractional shortening (FS) were determined based on M-mode measurements.

### 1.4 AAV-Production

The AAV genome plasmid pds-TnT-mWIF-1 was generated via amplification of the WIF-1 sequence from murine cDNA with the primers WIF-1 NheI fwd:

(SEQ ID NO: 2)
5′TCAGTCGCTAGCGCCACCATGGCTCGGAGAAGAGC-3′
and
WIF-1 BsrGI rev:
(SEQ ID NO: 3)
5′-TCAGTCTGTACAGGGTTCACCAGATGTAATTGG-3′

(restriction sites NheI and BsrGI underlined; KOZAK sequence bold) following subcloning to the vector pCR®-Blunt (ThermoFisher Scientific). The correct sequence of the WIF-1 cDNA was confirmed by sequencing following cloning to a self-complementary AAV-genome plasmid via restriction with NheI and BsrGI. AAV vector production and purification was done using standard procedures. To purify the AAV, cell lysate Iodixanol step gradient centrifugation was used, as described elsewhere (Müller OJ 2006). AAV9 pseudotyped vectors were generated by co-transfection using the two plasmid system, which consists of helper plasmid pDP9rs and either genome plasmid pdsTnT-rluc or—pds-TnT-mWIF-1. pdsTnT-rluc contained a Renilla luciferase reporter gene under control of the human troponin-T promoter and pds-TnT-mWIF-1 contained the murine cDNA of WIF-1.

### 1.5 Flow Cytometry and FACS-Sorting

Single-cell suspensions of infarcted hearts were obtained by mincing the tissue with fine scissors and digesting it with a solution containing collagenase I, collagenase XI, hyaluronidase (Sigma-Aldrich Chem GmbH, Taufkirchen, Germany), and DNase I (BD Biosciences, Heidelberg, Germany). 10×10⁶ cells were stained for flow cytometric analyses. Inflammatory monocytes were identified as

Lin⁻(CD90;B220;CD49b;NK1.1;Ly6G;Ter119);F4/80⁻;CD11c⁻;CD11b⁺;Ly6C^{hi}.
Neutrophils were identified as Lin⁺;CD11b⁺;F4/80⁻;CD11c⁻;Ly6C^{int}.
Flow cytometry was performed on FACS Verse (BD Biosciences, Heidelberg, Germany).
Data were analyzed using FlowJo v10 (FLOWJO, LLC, Ashland, USA).

For FACS-sorting, 10 animals underwent LAD ligation. Single cell suspensions of heart and bone marrow were pooled. Inflammatory monocytes from infarcted hearts and bone marrow cell suspensions were sorted on FACS ARIAII (BD Bioscience, Heidelberg, Germany). RNA of sorted inflammatory monocytes was isolated using AllPrep DNA/RNA Micro Kit (Qiagen, Hilden, Germany). FACS sorting was conducted three times.

### 1.6 RNAseq Analysis

RNA was quantified using a fragment analyzer (Advanced Analytical Technologies Inc., Heidelberg, Germany). 10 ng total RNA from sorted monocytes was used for library preparation. RNAseq libraries were generated using TrueSeq RNA Access Library Prep Kit (Illumina, San Diego, USA). Libraries were then clustered at a concentration of 8 pmol, and sequencing was performed on a HiSeq2000 (Illumina, San Diego, USA) sequencer.

### 1.7 Read Processing and Mapping

All reads were trimmed and quality clipped with Flexbar (Dodt et al. 2012). All remaining reads (>18 bp in length) were mapped against the murine 45S rRNA precursor sequence (BK000964.3) to remove rRNA contaminant reads. We used the mouse genome sequence and annotation (EnsEMBL 79) together with the splice-aware STAR read aligner (release 2.5.1b) (Dobin et al. 2013) to assemble our conditions-specific target transcriptomes. Subsequent transcriptome analyses on differential gene and transcript abundance were carried out with the cufflinks package (Trapnell et al. 2012).

### 1.8 Gene Enrichment Analysis

Gene set enrichment analyses were carried out within the R framework (Dobin et al. 2013) using topGO (for GeneOntology) and EGSEA (for MSigDB) packages.

### 1.9 Isolation of Neonatal Rat Ventricular Cardiomyocytes and Hypoxia

Neonatal rat ventricular cardiomyocytes (NRVCMs) were isolated as previously described (Hagenmueller et al. 2010). After plating, NRVCMs were allowed to adhere overnight. Cardiomyocytes were cultured under hypoxic conditions (1.5% O₂) for either four or 24 hours. Supernatant, RNA and proteins were harvested.

### 1.10 AdWIF1 Transfection of Cardiomyocytes

Premade WIF1 and control adenovirus was purchased from Applied Biological Materials Inc. (Richmond, Canada). Adenovirus was amplified according to the manufacturer's instructions. Cardiomyocytes were transfected according to manufacturer's instructions. WIF1 overexpression was allowed to establish for 48 hours. Transfected cardiomyocytes were than cultured under hypoxic conditions as described above (see 1.9).

### 1.11 Isolation and Stimulation of PBMC-Derived Macrophages

Adult rats were euthanized using a high dose of pentobarbital. Blood was collected by cardiac puncture into EDTA-coated sample tubes. Mononuclear cells were separated using Histopaque 1083 (Sigma-Aldrich Chemie GmbH, Taufkirchen, Germany) according to manufacturer's instructions. Monocytes/macrophages were allowed to adhere to the cell culture dish for two hours. Non-adherent mononuclear cells were discarded. Macrophages were stimulated with supernatant of hypoxic cardiomyocytes for four hours.

### 1.12 Western Blot Analysis

Protein lysates of primary cells and heart tissue were prepared using RIPA buffer supplemented with phosphatase/proteinase inhibitors (Cell Signaling Technology, Danvers, USA). Protein concentrations were measured using BCA assay (ThermoFisher Scientific, Waltham, USA). Equal amounts of protein were separated onto four 15% SDS-PAGE gradient gels (Bio-Rad Laboratories GmbH, München, Germany) and transferred to PVDF membranes (Merck Chemicals GmbH, Darmstadt, Germany). The following primary and HPR-conjugated secondary antibodies were used: monoclonal mouse anti-Active-β-Catenin (anti ABC), clone 8E7 (Merck Millipore, Darmstadt, Germany); monoclonal rabbit anti-JNK1+JNK2+JNK3 [EP1597Y] (phosphor Y223+Y185+Y185) (Abcam, Cambridge, UK); monoclonal rabbit anti-WIF1 [EPR9385] (Abcam, Cambridge, UK); monoclonal rabbit anti-GAPDH [EPR16891] (Abcam, Cambridge, UK); polyclonal goat anti-p-JNK (Santa Cruz, Heidelberg, Germany); monoclonal mouse anti-actinin (Sigma Aldrich, Munich, Germany); HRP-conjugated polyclonal rabbit anti-mouse IgG (Abcam, Cambridge, UK); HRP-conjugated goat anti-rabbit IgG (Abcam, Cambridge, UK). Proteins were visualized using Amersham ECL Western blotting detection reagents (GE Healthcare Europe GmbH, Freiburg, Germany). Images were captured using Peqlab Fusion FX (Peqlab Inc., Erlangen, Germany). Protein expression was analyzed using ImageJ. Protein bands were normalized to GAPDH (respectively actin) loading control for quantification.

### 1.13 Quantitative Real-Time PCR

Total RNA was isolated from primary cells using Trizol reagent (ThermoFisher Scientific, Waltham, USA) following manufacturer's instructions. RNA was reverse transcribed using Revert Aid First Strand cDNA Synthesis Kit (ThermoFisher Scientific, Waltham, USA). RT-qPCR was performed using SYBR Green (Bio-Rad Laboratories GmbH, München, Germany) according to manufacturer's instructions. Gene expression was normalized to HPRT. The following primers were used:

WIF1:
(SEQ ID NO: 4)
TTCTTTAAAACATGTCAACAAGCTG (fwd),
(SEQ ID NO: 5)
ACAAAAGCCTCCATTTCGAC (rev);
HPRT:
(SEQ ID NO: 6)
GTCAACGGGGGACATAAAAG (fwd),
(SEQ ID NO: 7)
TGCATTGTTTTACCAGTGTCAA (rev);
IL1β:
(SEQ ID NO: 8)
TCGTGCTGTCTGACCCATGT (fwd),
(SEQ ID NO: 9)
ACAAAGCTCATGGAGAATACCACT (rev);
IL6:
(SEQ ID NO: 10)
AACTCCATCTGCCCTTCAGGAACA (fwd),
(SEQ ID NO: 11)
AAGGCAGTGGCTGTCAACAACATC (rev).

### 1.14 Immunofluorescent WIF1 Staining

Paraffin-embedded heart tissue sections from patients who died of acute myocardial infarction were provided by the tissue bank of the National Center for Tumor Diseases (NCT, Heidelberg, Germany) in accordance with its regulations and the approval of Heidelberg University's ethics committee. Paraffin-embedded mouse and human heart tissue sections were deparaffinized using xylol and rehydrated in decreasing ethanol concentrations. Antigen retrieval was performed using citrate buffer for 15 minutes at 90° C. Sections were stained with polyclonal goat anti-rabbit WIF1 (Abcam, Cambridge, UK). WIF1 antibodies were fluorescently labeled using goat anti-rabbit Alexa Fluor 694 (Abcam, Cambridge, UK). Images were acquired on an Axio Observer (Carl Zeiss Microscopy GmbH, Jena, Germany).

### 1.15 Statistics

Statistical analyses were performed using GraphPad Prism 6 (GraphPad Software Inc., La Jolla, USA). Data are represented as mean±SD. Differences between two groups were analyzed by student's t test. Differences between more than two groups were analyzed by one-way ANOVA followed by Sidak post hoc analysis. P<0.05 was considered to be statistically significant.

### 1.16 Study Approval

Animal studies were approved by the regulatory authorities (Regierungspräsidium Karlsruhe of the state of Baden-Württemberg/Germany, 35-9185.81/G27/14). Human tissue samples were provided by the tissue bank of the National Center for Tumor Diseases (NCT, Heidelberg, Germany) in accordance with the regulations of the tissue bank and the approval of the ethics committee of Heidelberg University (Project number 1974).

### 1.17 Analysis of Effect of LGK-974 on Cardiomyocytes In Vivo

### Reagents:

Dulbecco's Phosphate Buffered Saline (DPBS), ThermoFisher Scientific

Dulbecco's Modified Eagle Medium: Nutrient Mixture F-12 (DMEM/F12),

ThermoFisher Scientific

Penicillin Streptomycin (pen/strep), ThermoFisher Scientific

Fetal Bovine Serum (FBS), ThermoFisher Scientific

Dimethylsulfoxid (DMSO), Sigma Aldrich

LGK-974, Selleckchem

10× Radioimmunoprecipitation assay (RIPA) buffer, Abcam

BCA Protein Assay, ThermoFisher Scientific

Mini-PROTEAN TGX Gels, Biorad

10×TG (transfer buffer), Biorad

10×TGS (running buffer), Biorad

Anti-Active-Beta-Catenin, Millipore

Anti-GAPDH, Abcam

Anti-Caspase-3, Cell Signaling

Anti-cleaved-Caspase-3, Abcam

Complete medium: DMEM/F12 supplemented with 1% pen/strep and 10% FBS
Starvation medium: DMEM/F12 supplemented with 1% pen/strep
LGK-974 stock solution: 10 mM (in DMSO)

Neonatal rat ventricular cardiomyocytes (NRVCM) were isolated as described above in 1.9. Isolated NRVCMs were resuspended in complete medium. 2×10⁶ NRVCMs/well were seeded in 6-well plates. Cells were allowed to adhere overnight.

Cells were washed 1× with DPBS to remove non-adherent and dead cells. NRVCMs were pre-incubated with 10 μM LGK-974 (or with an equal amount DMSO as a control) in starvation medium for 4 h.

LGK-974 and DMSO supplemented starvation medium was refreshed. Cells were cultured under normoxic or hypoxic (1.5% 02) conditions for 24 h.

Proteins of cultured NRVCMs were isolated using RIPA buffer according to manufacturer's instructions.

Protein concentrations were determined using BCA assay according to manufacturer's instructions.

15 μg protein/were loaded on precast 4-15% Mini-Protein TGX Gels. Electrophoresis and western blotting was performed using Bio-Rad mini protean blotting system (according to manufacturer's instructions).

Blots were analyzed for active-beta-catenin, (total) caspase-3, and cleaved-caspase-3 expression. Protein expression was normalized to GAPDH.

1.18 Analysis of In Vivo Effect of LGK-974 on Monocyte Inflammatory Processes after MI

Female C57BL/6N mice at the age of 8-9 weeks underwent ischemia/reperfusion (I/R) surgery (ischemia was induced for 30 min). Animals received two doses of 3 mg/kg LGK-974 or vehicle control (DMSO) in citrate buffer via oral gavage (directly after surgery and 24 h after surgery). Blood was collected 24 h after surgery to evaluate initial infarct sizes. Animals with no infarction and very large infarcts (Troponin T levels>5000 pg/ml) were excluded from the study. FACS analysis was performed two days after I/R surgery.

Leukocytes were identified as CD45+.
Inflammatory monocytes were identified as CD45+;Lineage−;CD11b+;CD11c−;Ly6C+.

### 2. Results

### 2.1 the Microenvironment Orchestrates WNT Signaling in Inflammatory Monocytes

In order to assess how traveling leukocytes adapt to their surroundings during inflammation, we isolated inflammatory Ly6C^{hi} monocytes from mice three days after myocardial infarction. RNA-seq analyses revealed that cardiac necrosis results in a diverse transcriptional response in inflammatory monocytes sorted from the bone marrow (BM), blood and heart. We observed differential expression of 1482 genes in Ly6C^{hi} monocytes from these locations (data not shown). Of note, we found similar expression patterns in Ly6C^{hi} monocytes' transcriptome. A great proportion of the transcriptome was associated with WNT signaling (data not shown).

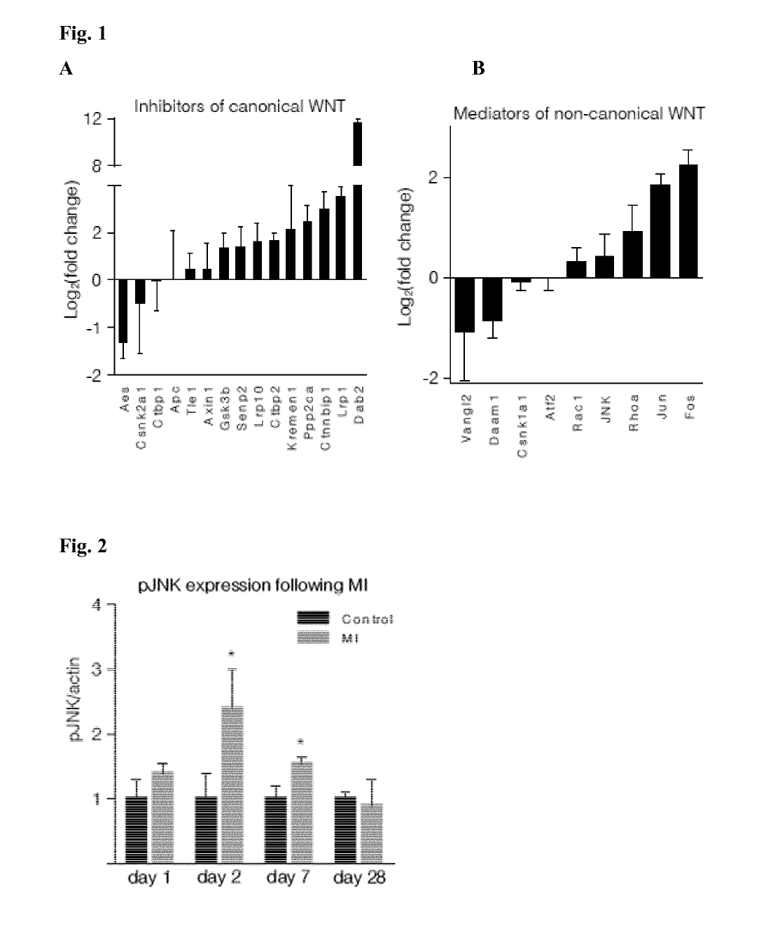

In addition, gene set enrichment analysis (GSEA) revealed highly and significantly increased genes bearing AP-1 and LEF1 binding sites and differential expression of 39 WNT associated genes (data not shown). LEF1 and AP-1 (Bengoa-Vergniory et al. 2015) have been described to regulate WNT-induced gene expression of the canonical and non-canonical WNT signaling pathways, respectively. To further elucidate the regulation of different WNT signaling pathway branches in Ly6C^{hi} monocytes from different regions, we further analyzed components of both the canonical and non-canonical WNT pathways. Comparing Ly6C^{hi} monocytes isolated from infarcted myocardium and bone marrow showed increased numbers of canonical WNT inhibitors (i.e. members of the β-catenin destruction complex), whereas canonical WNT signaling mediators mostly decreased. By contrast, non-canonical WNT/PCP pathway mediators and target genes were upregulated in monocytes isolated from the infarcted heart compared to those from the bone marrow (FIGS. 1A and 1B).

To further validate the role of the non-canonical WNT/PCP pathway, we evaluated phosphorylated JNK (pJNK) expression levels in post-MI murine heart whole-tissue lysates. Two days after induced MI, Western blot analysis showed increased pJNK levels, which remained elevated until day seven post-MI, as compared to sham-operated animals (FIG. 2). In vitro, we found that hypoxic cardiomyocyte supernatant activates the non-canonical WNT pathway in macrophages through JNK phosphorylation. By contrast, the canonical WNT pathway was downregulated in the same macrophages, a result that corroborated the findings described above (data not shown).

### 2.2 WIF1 Increases in Hypoxic Cardiomyocytes and is Secreted in the Border Zone Following MI

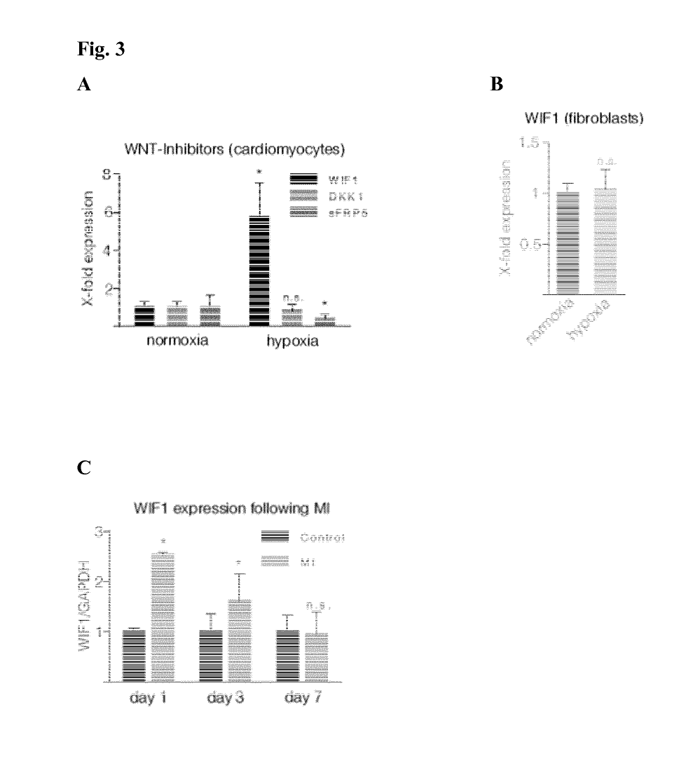

Since the local milieu seems to drive WNT regulation in accumulating monocytes, we evaluated several extracellular WNT antagonists in an in vitro MI model. In accordance with existing/previous research, we found WNT antagonists to be either unaffected or attenuated after hypoxia (e.g. DKK1, SFrp5; FIG. 3A). In contrast, the WNT antagonist WIF1 significantly increased in hypoxic cardiomyocytes but not in hypoxic fibroblasts (FIGS. 3A and B). In addition, WIF1 elevated at early time points (days 1 and 3 post-MI) but returned to baseline levels on day seven after cardiac injury (FIG. 3C). Immunofluorescent staining revealed WIF1 expression to be predominantly located in the border zones of infarcted hearts (data not shown). In addition, WIF1 was detected in tissue sections from human patients with acute MI in contrast to ischemic cardiomyopathy (data not shown).

2.3 Absence of WIF1 Results in Impaired Healing after MI

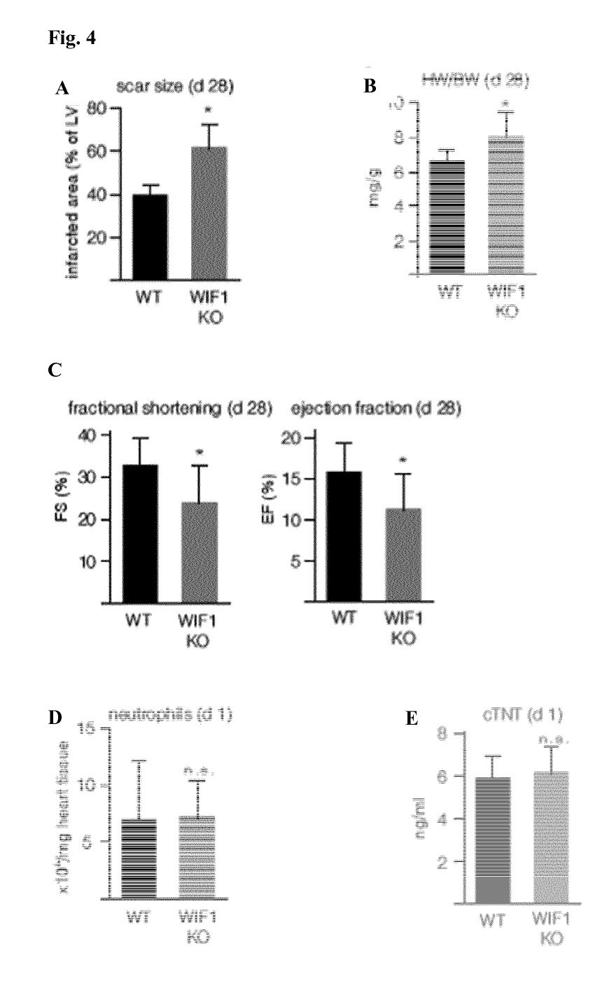

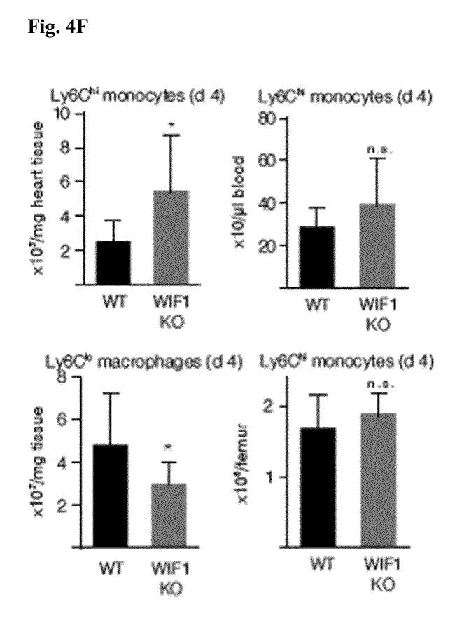

The altered WIF1 infarcted tissue and isolated cardiomyocytes encouraged us to further clarify WIF1's role in the post-MI healing process. We therefore induced MI in global WIF1 knockout animals and their WT littermates by permanent LAD ligation. WIF1 knockout mice developed normally (Kansara M 2009) and showed no cardiac-specific phenotype under basal conditions, as evaluated by echocardiography (data not shown). Four weeks after induced MI, masson trichrome staining revealed significantly increased infarct size in WIF1 KO animals, as compared to WT mice, (FIG. 4A). In addition, WIF1 KO animals showed higher heart weight/body weight ratio than their WT littermates (FIG. 4B). Echocardiographic analysis, moreover, revealed that WIF1 KO mice had developed severe cardiac dysfunction marked by significantly reduced fractional shortening (FS) and ejection fraction (EF), as compared to their WT littermates four weeks after MI (FIG. 4C).

Since ischemia triggers modulated WNT signaling in leukocytes, we evaluated the cellular inflammatory response in the presence and absence of WIF1. Flow cytometric analysis of mice hearts showed unaltered neutrophil numbers in WIF1 KO animals one day after induced MI, as compared to WT littermates (FIG. 4D). Additionally, infarct size, as measured by cardiac Troponin T (cTnT) levels in plasma samples, was unchanged in WIF1 KO mice and WT littermates one day after induced cardiac injury (FIG. 4E). In contrast, the monocyte/macrophage response was critically altered four days after MI: the hearts of WIF1 KO animals had significantly more inflammatory Ly6C^{hi} monocytes (FIG. 4F (top left graph)) and fewer reparative Ly6C^{lo} macrophages than hearts from WT mice (FIG. 4F (bottom left)). Inflammatory monocyte levels in the blood and bone marrow did not change (FIG. 4F (top right and bottom right)).

To evaluate whether the WIF1 in myeloid cells impacts the immune response following MI, we performed bone marrow transfers from WIF1 KO mice into WT recipients. FACS analysis of hearts on day four after MI showed no significant differences between the groups' numbers of Ly6C^{hi} monocytes and Ly6C^{lo} macrophages (not shown).

2.4 Cardiomyocyte-Specific WIF1 Overexpression Improves Healing after MI

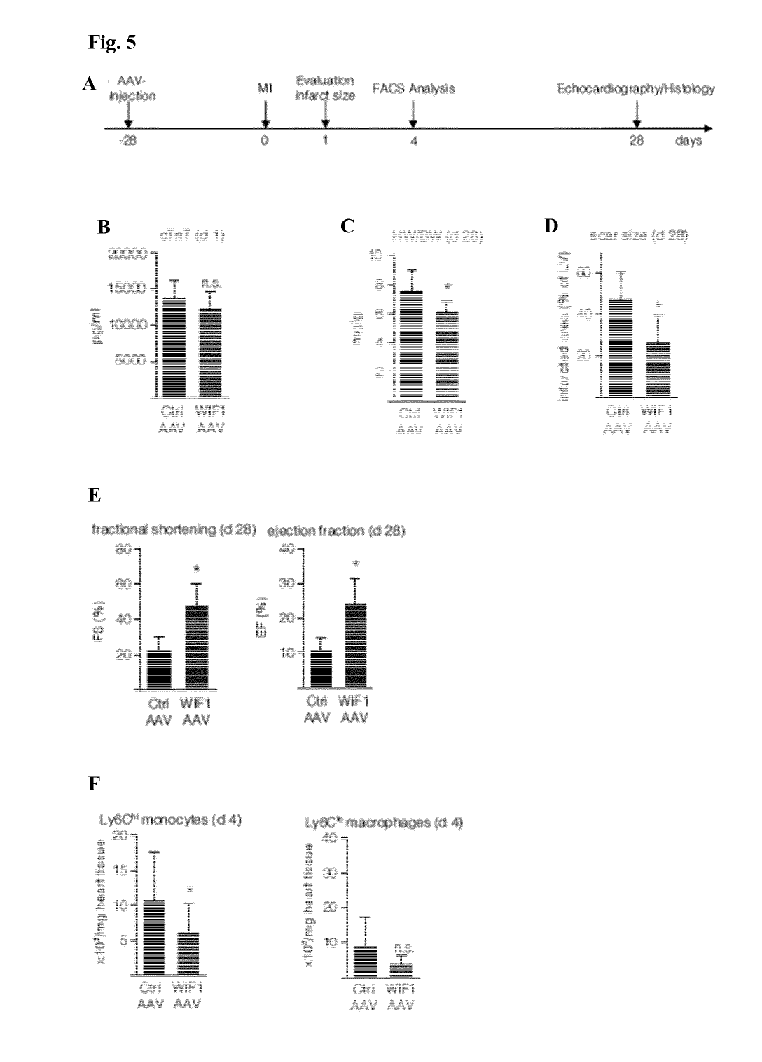

Because the absence of WIF1 impedes the post-MI healing process, we tested if WIF1 overexpression could enhance myocardial healing and improve heart function following cardiac injury. We therefore injected WT animals with either cardiotropic WIF1-AAV9 or a control LUC-AAV9 vector (under the control of a troponin T promoter) before inducing MI (see FIG. 5A for timeline). To evaluate if cardiomyocyte-specific WIF1 overexpression alters infarct sizes at early time points, we measured cTnT levels in blood plasma 1 day after inducing myocardial infarction. We observed no differences between the groups (FIG. 5B). 28 days after MI, WIF1-overexpressing animals had reduced heart weight/body weight ratios (FIG. 5C). In addition, masson trichrome staining revealed that AAV9-WIF1 injected animals had significantly smaller scar sizes four weeks after induced MI (FIG. 5D). On day 28 after MI, we evaluated cardiac function by using echocardiography to measure FS and EF percentages. WIF1 overexpression led to significantly improved FS and EF compared to control animals, a result that indicated ameliorated cardiac healing after MI (FIG. 5E). In contrast to previous reports (Lu et al. 2013), we did not detect differences in cardiac function in WIF1-overexpressing vs. control animals (data not shown).

2.5 Cardiomyocyte-Specific WIF1 Overexpression Attenuates Cardiac Inflammation after MI

Since we observed significantly different post-MI monocytic responses in WIF1 KO animals compared to WT mice, we next tested if cardiomyocyte-specific WIF1 overexpression impacts monocyte/macrophage numbers and the inflammatory response following MI. A robust heart-specific WIF1 overexpression was achieved (not shown) and led to reduced Ly6C^{hi} monocyte numbers in the heart four days after induced MI (FIG. 5F (left graph)). The number of reparative Ly6C^{lo} macrophages, however, did not significantly differ (FIG. 5F (right graph)).

### 2.6 WIF1 Ameliorates Monocyte/Macrophage Inflammatory Processes by Inhibiting Non-Canonical WNT Signaling

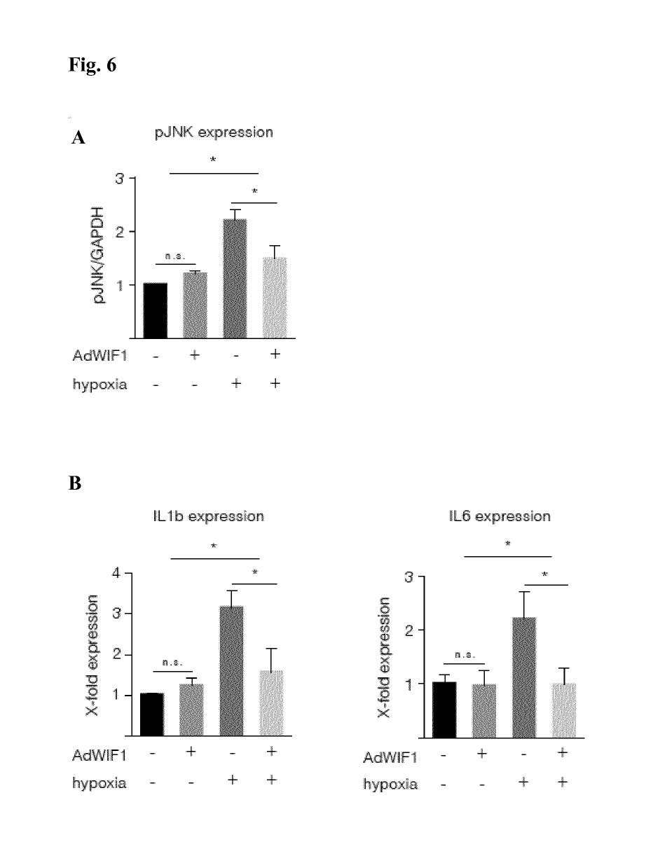

Elegant binding analysis has previously shown that WIF1 interacts with the non-canonical WNT pathway's extracellular activators (Malinauskas et al. 2011). To evaluate if cardiomyocyte-derived WIF1 affects myeloid cell activation, we induced WIF1 overexpression in isolated cardiomyocytes and exposed the cells to hypoxia. Supernatant of hypoxic cardiomyocytes activated JNK phosphorylation in isolated monocytes/macrophages. This activation was significantly reduced after transfer of supernatant from hypoxic cells that overexpressed WIF1 (FIG. 6A). In addition, monocytes/macrophages treated with supernatant of WIF1-overexpressing cardiomyocytes showed reduced transcription levels of pro-inflammatory cytokines IL1β and IL6, as compared to control cells (FIG. 6B), thereby indicating that WIF1 reduces pro-inflammatory monocyte/macrophage activation by hypoxic cardiomyocytes.

### 2.7 LGK-974 Inhibits Activation of the WNT Signaling Pathway in Stressed/Hypoxic Cardiomyocytes.

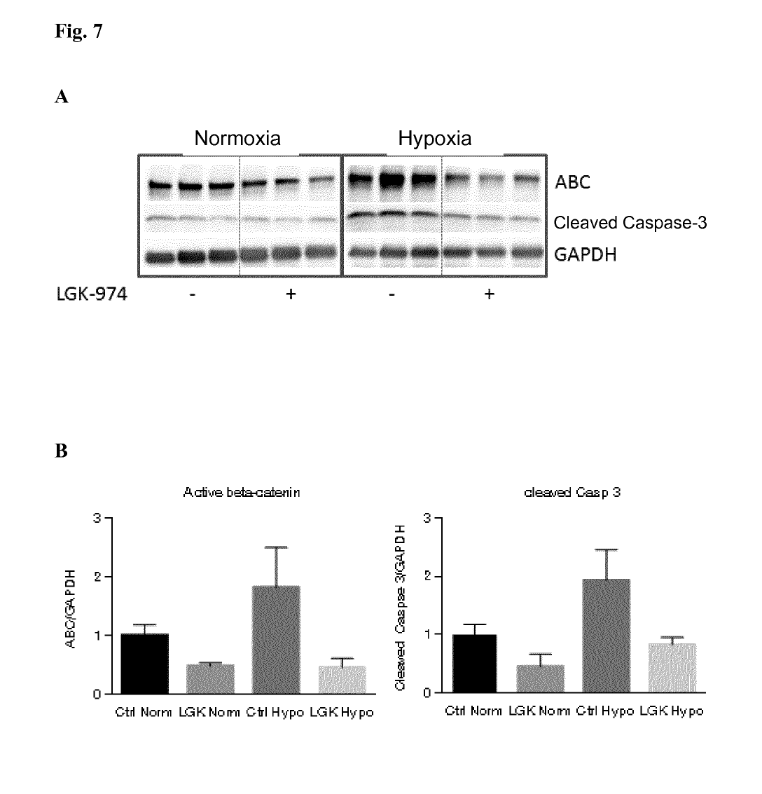

LGK-974 is a potent and selective Porcn inhibitor, which can prevent secretion of WNT proteins. Our in vitro data show that—through the inhibition of Porcn by LGK-974—the activation of the WNT signaling pathway is inhibited in stressed/hypoxic cardiomyocytes and that the apoptosis of cardiomyocytes decreases after pathological stimulus (see FIGS. 7A and 7B, right diagram, reduced expression of cleaved caspase-3, which is involved in apoptosis). In addition, it was found that stressed cardiomyocytes treated with LGK-974 stimulate immune cells to a lesser extent than non-treated stressed cardiomyocytes. This is evident both from a reduced activation of WNT (reduced expression of pATF2, data not shown; reduced expression of active-beta catenin, which is signal transducer of the WNT pathway, see FIGS. 7A and 7B left diagram) and by reduced expression of pro-inflammatory cytokines in immune cells (data not shown).

2.8 LGK-974 Reduces Monocyte Inflammatory Processes after MI.

Experimental details of this experiment are given above in section 1.18. In principle, this experiment is comparable to the experiment relating to cardiomyocyte-specific WIF1 overexpression described in sections 2.4 and 2.5 above.

However, in the experiment described in sections 2.4 and 2.5, myocardial infarction (MI) was induced by permanent ligation of the left anterior descending (LAD) coronary artery. In the following experiment, the LAD coronary artery was only temporarily ligated (i.e. for 30 min) by way of ischemia/reperfusion (I/R) surgery. I/R surgery mirrors the clinical situation in which a patient with myocardial infarction receives surgical treatment in which the occluded artery is reopened by means of a catheter. Both in said clinical situation and in the model of I/R surgery, a temporary occlusion of a cardiac artery leads to the death of cardiac tissue and to the induction of a strong immune reaction.

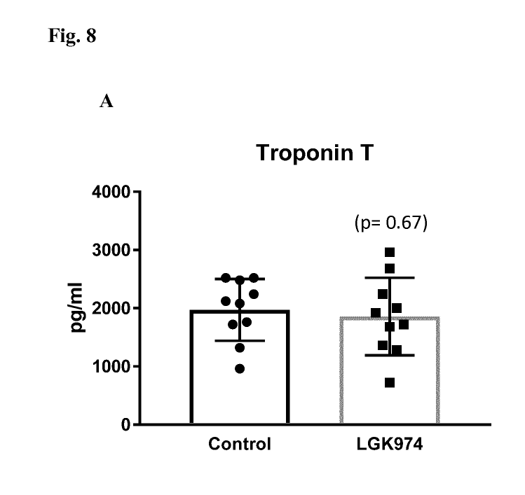

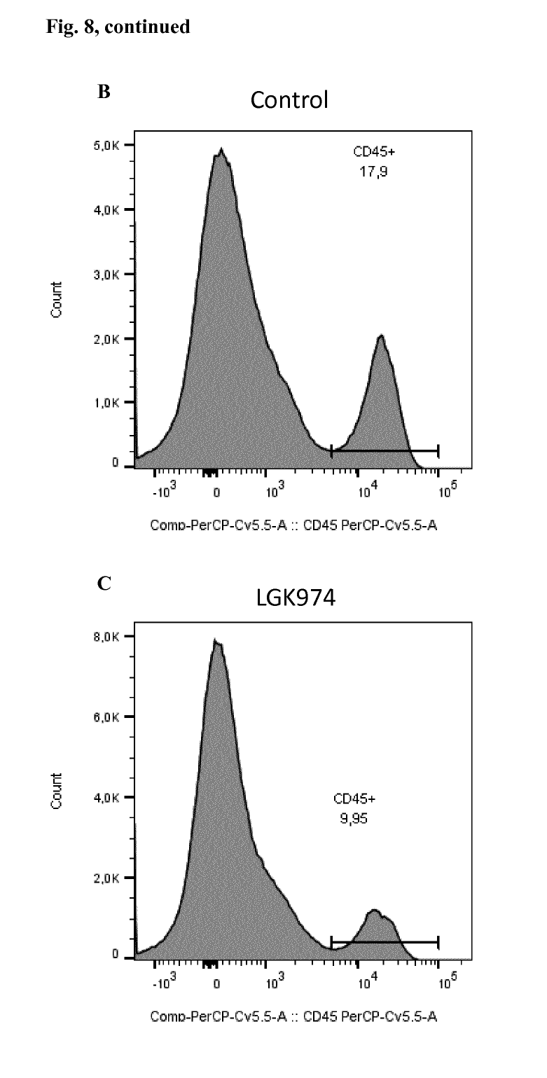

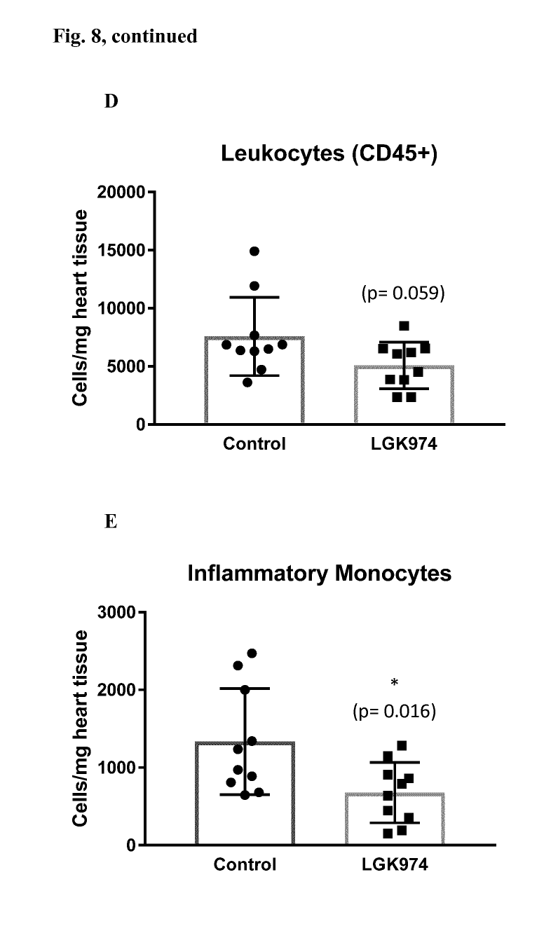

Troponin T levels were determined in blood collected 24 h after surgery to evaluate initial infarct sizes (see FIG. 8A). Animals treated with LGK-974 and control animals had the same troponin T levels, which demonstrates that any differences observed were not caused by differences in infarction size but were due to the treatment with LGK-974.

The overall immune response is reduced in LGK-974-treated animals as compared to control animals, as can be concluded from the lower number of leukocytes per mg heart tissue in LGK-974-treated animals (see FIG. 8C and FIG. 8D, right column) as compared to control animals (see FIG. 8B and FIG. 8D, left column). The p-value in this comparison was 0.059 (see FIG. 8D).

In addition, the infiltration of inflammatory monocytes into the infarcted heart was studied. Inflammatory monocytes were identified as CD45+;Lineage−;CD11b+;CD11c−;Ly6C+. A significant reduction of inflammatory monocytes in the infarcted heart was observed in LGK-974-treated mice as compared to the control group (see FIG. 8E; p=0.016).

Without wishing to be bound by theory, the inventors believe that excessive inflammatory reactions (e.g. by inflammatory monocytes) have a negative impact on the healing process after MI and may cause additional tissue damage. The aforementioned results show that inflammatory reactions can be modulated by LGK-974, which will improve healing after MI and will have positive effects on heart function (see also echocardiographic results of mice with cardiomyocyte-specific WIF1 overexpression in section 2.4 above).

### 3. Discussion

Acute myocardial infarction causes a sterile, systemic inflammatory response. Ischemia and necrosis induce complement system activation, damage-associated molecular pattern molecules (DAMPs) release and integrin activation on endothelial cells, thereby resulting in myeloid cell invasion at the injury site (Leuschner et al. 2012; Frangogiannis 2014). Our findings suggest that upon accumulation, leukocytes are locally adjusted by WNT modulators actively secreted by cardiomyocytes. Previous reports have shown WNT signaling to be crucial following MI and ischemia reperfusion injury (Daskalopoulos et al. 2013; Bao et al. 2015; Nakamura et al. 2015); however, WNT signaling's precise role in regulating immune response—especially in the context of MI—is not yet understood. The present inventors have shown, for the first time, that the microenvironment is an important determinant of WNT signaling in the immune cell activation that leads to phenotypic changes in monocytes.

Our RNAseq analysis shows that non-canonical WNT signaling is augmented in Ly6C^{hi} monocytes isolated from the heart, but not the blood or bone marrow, in mice with myocardial infarction, thereby illustrating the microenvironment's interaction with recruited monocytes. We also found the canonical WNT pathway is suppressed in Ly6C^{hi} monocytes at the site of inflammation. It was previously reported that canonical WNT signaling is activated in mononuclear bone marrow cells after MI (Assmus et al. 2012; Hermans et al. 2012). We found more components of the non-canonical WNT pathway and intracellular canonical WNT pathway inhibitors in monocytes isolated from the heart than in monocytes from the bone marrow. These data suggest that the non-canonical WNT pathway is activated and predominant in monocytes in the infarcted heart. Accordingly, non-canonical WNT signaling increases in whole-heart tissue during the first week after MI, a phase characterized by the presence of leukocytes in the heart. In addition, our in vitro data suggest that troubled cardiomyocytes can activate non-canonical WNT signaling in monocytes directly, since monocyte/macrophage stimulation with hypoxic cardiomyocyte supernatant led to increased JNK phosphorylation and simultaneously decreased canonical WNT pathway. Taken together, these findings support the idea that the non-canonical WNT signaling pathway is involved in inflammatory processes following myocardial infarction. Furthermore, these data show paracrine communication between cardiomyocytes and monocytes/macrophages has significant impact on both WNT signaling activity at the site of inflammation and the quality of recuperation after ischemic injury. This interaction appears to localize in the infarct border zone, which is both where most leukocytes accumulate in the injured myocardium and a crucial location for ongoing remodeling processes (Leuschner et al. 2010; Leuschner et al. 2011).

The inflammatory response following myocardial infarction is integral to adequate infarct healing. Neutrophils, Ly-6C^{hi} monocytes, and Ly6C^{lo} macrophages are responsible clearing necrotic tissue and developing a solid scar. However, an exaggerated inflammatory response aggravates infarct healing and may promote heart failure (Nahrendorf et al. 2007; Geissmann et al. 2010; Nahrendorf et al. 2010). Our research shows the extracellular WNT antagonist WIF1 to be a vital modulator of an adequate inflammatory process following cardiac injury. In addition, WIF1's presence in human heart tissue samples indicates its potential relevance in patients with myocardial infarction. In contrast to previous reports that suggest WNT antagonists decrease during MI (Bao et al. 2015; Nakamura et al. 2015), we surprisingly found elevated WIF1 persisted during the inflammatory phase of myocardial healing. Although we cannot exclude the possibility that invading cells import additional WIF1, our in vitro studies show increased WIF1 in cardiomyocytes but not in cardiac fibroblasts after hypoxia. Further, we observed in vivo that bone marrow transfer of WIF1 KO cells into WT mice did not alter the immune response. These results indicate that cardiomyocytes are the crucial source of WIF1 after MI. The fact that cardiomyocyte-specific WIF1 overexpression was sufficient to modulate the monocyte response supports this hypothesis.

Genetic WIF1 deficiency led to larger infarcts, reduced heart function, and increased inflammation. Augmented WNT signaling due to the absence of other WNT inhibitors or overexpression of WNT activators has been associated with increased cardiomyocyte death (van de Schans et al. 2008; Bergmann 2010; Daskalopoulos et al. 2013). However, our experiments with WIF1 KO and WT animals showed no difference in initial infarct sizes between the groups. We therefore hypothesized that reduced left ventricular function in WIF1 KO animals 28 days after myocardial infarction resulted from an altered inflammatory response. Since transplanting WIF1 KO bone marrow cells into WT recipient mice had no effect on the inflammatory response following MI, we concluded that WIF1 does not act on cardiomyocytes but rather on the infiltrating immune cells in a paracrine manner. Yet WIF1 does not seem to alter the general inflammatory response. We detected no difference in neutrophil numbers between WIF1KO and WT animals, whereas monocyte and macrophage numbers were significantly altered. We therefore concluded that WIF1 activity is cell-type specific and may fine-tune WNT signaling at the site of inflammation. Given the increased numbers of proinflammatory Ly6C^{hi} monocytes and reduced reparative Ly6Clo macrophages in WIF1 KO mice after MI, WIF1 may either accelerate the resolution of inflammation after MI or prevent the continuation of ongoing proinflammatory stimuli.

Finally, we investigated WIF1's protective potential in the context of myocardial infarction. Cardiomyocyte-specific WIF1 overexpression led to significantly fewer inflammatory monocytes in the heart after MI while reparative macrophage numbers remained unaltered. This result challenges the hypothesis that WIF1 and non-canonical WNT pathway inhibition might be involved in the Ly6C^{hi} monocytes' differentiation into Ly6C^{lo} macrophages. Stimulating macrophages with supernatant of hypoxic cardiomyocytes that overexpress WIF1, however, not only led to reduced non-canonical WNT signaling activation but also reduced pro-inflammatory cytokine mRNA levels. These findings clearly indicate that WIF1 interferes with non-canonical WNT signaling in monocytes and macrophages and reduces pro-inflammatory activation. AAV-mediated WIF1 overexpression supports myocardial healing and improves cardiac function after MI. Adeno-associated virus-based gene therapy has been approved for clinical practice (Flotte 2013). Nevertheless, alternative WIF1 modulation and administration are also contemplated within the scope of the present invention. In particular, the inventors have also studied the effect of LGK-974 (a small molecule) on stressed/hypoxic cardiomyocytes in vitro and have found that LGK-974 has an immunomodulatory effect on stressed cardiomyocytes. In addition, the inventors have studied the effect of LGK-974 on inflammatory processes in heart tissue of mice after myocardial infarction (induced by I/R surgery) and have found that LGK-974 reduces infiltration of inflammatory monocytes into the heart. These results show that LGK-974 is a suitable compound for treating myocardial infarction, especially for reducing tissue damage, for reducing infarction scars, for improving cardiac function and/or for preventing congestive heart failure after myocardial infarction.

### SEQUENCE LISTING FREE TEXT INFORMATION

SEQ ID NO: 1 wound-homing sequence
SEQ ID NO: 2 primer WIF-1 NheI fwd
SEQ ID NO: 3 primer WIF-1 BsrGI rev
SEQ ID NO: 4 primer WIF1 (fwd)
SEQ ID NO: 5 primer WIF1 (rev)
SEQ ID NO: 6 primer HPRT (fwd)
SEQ ID NO: 7 primer HPRT (rev)
SEQ ID NO: 8 primer IL1beta (fwd)
SEQ ID NO: 9 primer IL1beta (rev)
SEQ ID NO: 10 primer IL6 (fwd)
SEQ ID NO: 11 primer IL6 (rev)

### REFERENCES

Assmus, B., M. Iwasaki, et al. (2012). “Acute myocardial infarction activates progenitor cells and increase Wnt signalling in the bone marrow.” Eur Heart J. 33(15): 1911-1919.
Bao, M. W., Z. Cai, et al. (2015). “Dickkopf-3 protects against cardiac dysfunction and centricular remodelling following myocardial infarction.” Basic Res Cardiol 110(3): 25.
Bengoa-Vergniory, N. and R. M. Kypta (2015). “Canonical and noncanonical Wnt signaling in neural stem/progenitor cells.” Cell Mol Life Sci 72(21): 4157-4172.
Bergmann, M. W. (2010). “WNT signaling in adult cardiac hypertrophy and remodeling: lessons learned from cardiac development.” Circ Res 107(10): 1198-1208.
Christia, P. and N. G. Frangogiannis (2013). “Targeting inflammatory pathways in myocardial infarction.” Eur J Clin Invest 43(9): 986-995.
Clevers, H. and R. Nusse (2012). “Wnt/β-catenin signaling and disease.” Cell 149(6): 1192-1205.
Daskalopoulos, E. P., K. C. M. Hermans, et al. (2013). “Targeting the Wnt/frizzled signaling pathway after myocardial infarction: A new tool in the therapeutic toolbox?” Trends Cardiovasc Med 23(4): 121-127.
Dobin, A., C. Davis, et al. (2013). “STAR: ultrafast universal RNA-seq aligner.” Bioinformatics 29(1): 15-21.
Dodt, M., J. T. Roehr, et al. (2012). “FLEXBAR-Flexible Barcode and Adapter Processing for Next-Generation Sequencing Platforms.” Biology (Basel) 1(3): 895-905.
Eisenberg, L. M. and C. A. Eisenberg (2006). “Wnt signal transduction and the formation of the myocardium.” Dev Biol 293(2): 305-315.
Flotte, T. R. (2013). “Birth of a new therapeutic platform: 47 years of adeno-associated virus biology from virus discovery to licensed gene therapy.” Mol Ther 21(11): 1976-1981.
Frangogiannis, N. G. (2014). “The inflammatory response in myocardial injury, repair, and remodelling.” Nat Rev Cardiol 11(5): 255-265.
Geissmann, F., M. G. Manz, et al. (2010). “Development of monocytes, macrophages and dendritic cells.” Science 327(5966): 656-661.
Hagenmueller, M., P. Malekar, et al. (2010). “Depletion of mammalian target of rapamycin (mTOR) via siRNA mediated knockdown leads to stabilization of beta-catenin and elicits distinct features of cardiomyocyte hypertrophy.” FEBS Lett 584(1): 74-80.
He, S., B. G. Chousterman, et al. (2015). “Lp-PLA2 Antagonizes Left Ventricular Healing After Myocardial Infarction by Impairing the Appearance of Reparative Macrophages.” Circ Heart Fail 8(5): 980-987.
Hermans, K. C., E. P. Daskalopoulos, et al. (2012). “Interventions in Wnt signaling as a novel therapeutic approach to improve myocardial infarct healing.” Fibrogenesis Tissue Repair 5(1): 16.
Hilgendorf, I., L. M. Gerhardt, et al. (2014). “Ly-6Chigh monocytes depend on Nr4a1 to balance both inflammatory and reparative phases in the infarcted myocardium.” Circ Res 114(10): 1611-1122.
Hsieh, J. C., L. Kodjabachian, et al. (1999). “A new secreted protein that binds to Wnt proteins and inhibits their activities.” Nature 398(6726): 431-436.
Kansara M, T. M., Kodjabachian L, Sims N A, Trivett M K, Ehrich M, Dobrovic A, Slavin J, Choong P F, Simmons P J, Dawid I B, Thomas DM (2009). “Wnt inhibitory factor 1 is epigenetically silenced in human osteosarcoma, and targeted disruption accelerates osteosarcomagenesis in mice.” J Clin Invest 119(4): 837-851.
Kawano, Y. and R. Kypta (2003). “Secreted antagonists of the Wnt signaling pathway.” J Cell Sci 116(13): 2627-2634.
Kim, J., W. Chang, et al. (2012). “Wnt5a activates THP-1 monocytic cells via a β-catenin-independent pathway involving JNK and NF-κB activation.” Cytokine 60(1): 242-248.
Koval, A., V. Purvanov, et al. (2011). “Yellow submarine of the WNT/Frizzled signaling: submerging from the G protein harbor to the targets.” Biochem Pharmacol 82(10): 1311-1319.
Leuschner, F., P. Dutta, et al. (2011). “Therapeutic siRNA silencing in inflammatory monocytes in mice.” Nat Biotechnol 29(11): 1005-1010.
Leuschner, F., P. Panizzi, et al. (2010). “Angiotensin-converting enzyme inhibition prevents the release of monocytes from their splenic reservoir in mice with myocardial infarction.” Circ Res 107(11): 1364-1373.
Leuschner, F., P. J. Rauch, et al. (2012). “Rapid monocyte kinetics in acute myocardial infarction are sustained by extramedullary monocytopoiesis.” J Exp Med 209(1): 123-137.
Lu, D., W. Dong, et al. (2013). “WIF1 causes dysfunction of heart in transgenic mice.” Transgenic Res 22(6): 1179-1189.
Malinauskas, T., A. R. Radu Aricescu, et al. (2011). “Modular mechanism of Wnt signaling inhibition by Wnt inhibitory factor 1.” Nat Struct Mol Biol 18(8): 886-893.
Müller O J, L. B., Pleger S T, Grimm D, Franz W M, Katus H A, Kleinschidt J A (2006). “Improved cardiac gene transfer by transcriptional and transductional targeting of adeno-associated viral vectors.” Cardiovasc Res 70(1): 70-78.
Nahrendorf, M., M. K. Pittet, et al. (2010). “Monocytes: Protagonists of infarct inflammation and repair after myocardial infarction.” Circulation 121(22): 2437-2445.
Nahrendorf, M., F. K. Swirski, et al. (2007). “The healing myocardium sequentially mobilizes two monocyte subsets with divergent and complementary functions.” JEM 204(12): 3037-3047.
Nakamura, K., S. Sano, et al. (2015). “Secreted frizzled-related protein 5 diminishes cardiac inflammation and protects the heart from ischemia reperfusion injury.” J biol Chem (693937/R1).
Pereira, C., D. J. Schaer, et al. (2008). “WntSA/CaMKII Signaling Contributes to the Inflammatory Response of Macrophages and Is a Target for the Antiinflammatory Action of Activated Protein C and Interleukin-10.” Arterioscler Thromb Vasc Biol 28: 504-510.
Rauner, M., N. Stein, et al. (2012). “WNT5A is induced by inflammatory mediators in bone marrow stromal cells and regulates cytokine and chemokine production.” J Bone Miner Res 27(3): 575-585.
Saxena, A., I. Russo, et al. (2016). “Inflammation as a therapeutic target in myocardial infarction: learning from past failures to meet future challenges.” Transl Res 167(1): 152-166.
Swirski, F. K. and M. Nahrendorf (2013). “Leukocyte behavior in atherosclerosis, myocardial infarction, and heart failure.” Science 339(6116): 161-116.
Trapnell, C., A. Roberts, et al. (2012). “Differential gene and transcript expression analysis of RNA-seq experiments with TopHat and Cufflinks.” Nat Protoc 7(3): 562-578.
van de Schans, V. A., J. F. Smits, et al. (2008). “The Wnt/frizzled pathway in cardiovascular development and disease: friend or foe?” Eur J Pharmacol 585(2-3): 338-345.

1. A WNT antagonist for use in the treatment of myocardial infarction.

2. The WNT antagonist for use according to claim 1, wherein the WNT antagonist
(a) inhibits secretion of WNT,
(b) blocks binding of WNT to one or more WNT receptors, and/or
(c) inhibits production of WNT.

3. The WNT antagonist for use according to claim 2, wherein the binding of WNT to one or more WNT receptors is blocked by
binding of the WNT antagonist to WNT, and/or
binding of the WNT antagonist to said one or more WNT receptors.

4. The WNT antagonist for use according to claim 1, wherein the WNT antagonist is
(a) a small molecule, preferably LGK-974.
(b) an expression vector comprising a polynucleotide sequence encoding a protein that antagonizes the non-canonical WNT signalling pathway, or
(c) a protein.

5. The WNT antagonist for use according to claim 4, wherein the expression vector is an AAV vector, preferably an AAV9 vector.

6. The WNT antagonist for use according to claim 4, wherein the polynucleotide sequence is under control of a cardiomyocyte-specific promoter, preferably under control of the troponin T promoter.

7. The WNT antagonist for use according to claim 4, wherein the protein that antagonizes the non-canonical WNT signalling pathway is WNT inhibitory factor 1 (WIF1) or a fusion protein comprising WIF1.

8. The WNT antagonist for use according to claim 4, wherein the protein of paragraph (c) of claim 4 is WIF1 or a fusion protein comprising WIF1.

9. The WNT antagonist for use according to claim 8, wherein the fusion protein comprises a wound-homing sequence, preferably the wound-homing sequence CARSKNKDC (SEQ ID NO: 1).

10. A fusion protein comprising:
(a) WNT inhibitory factor 1 (WIF1), and
(b) a wound-homing sequence, preferably the wound-homing sequence CARSKNKDC (SEQ ID NO: 1).

11. An expression vector comprising a polynucleotide sequence encoding a protein that antagonizes the non-canonical WNT signalling pathway.

12. The expression vector according to claim 11, wherein the expression vector is an AAV vector, preferably an AAV9 vector.

13. The expression vector according to claim 11, wherein the polynucleotide sequence is under control of a cardiomyocyte-specific promoter, preferably under control of the troponin T promoter.

14. The expression vector according to claim 11, wherein the protein that antagonizes the non-canonical WNT signalling pathway is WNT inhibitory factor 1 (WIF1) or a fusion protein comprising WIF1.

15. The expression vector according to claim 14, wherein the fusion protein comprises a wound-homing sequence, preferably the wound-homing sequence CARSKNKDC (SEQ ID NO: 1).
